(19) United States
(12) Patent Application Publication
(10) Pub. No.: US 2002/0151785 A1
(43) Pub. Date: Oct. 17, 2002

(54) Mehtod and magnetic resonance tomography apparatus for preparing a data acquisition using previously obtained data acquisitions

(75) Inventors: Mike Mueller, Nuernberg (DE); Roland Schneider, Erlangen (DE)

Correspondence Address: SCHIFF HARDIN & WAITE, 6600 SEARS TOWER, 233 S WACKER DR, CHICAGO, IL, 60606-6473, US

(73) Assignee: Siemens Aktiengesellschaft

(21) Appl. No.: 10/119,321

(22) Filed: Apr. 9, 2002

(30) Foreign Application Priority Data
Apr. 11, 2001 (DE) 101 18 194.9

(51) Int. Cl. A61B 5/05

(52) U.S. Cl. 600/410

(57) ABSTRACT

In a method for preparing a magnetic resonance data acquisition procedure by using magnetic resonance images that have already been obtained and a magnetic resonance tomography apparatus using the method, imaging parameters of already-performed magnetic resonance data acquisitions are stored, magnetic resonance data acquisitions that have already been performed are simultaneously or subsequently graphically represented, and a selection possibility from among the already-obtained and graphically represented magnetic resonance, image is offered and graphically represented. The imaging parameters of a magnetic resonance image selected by a user are automatically transferred into the settings for imaging parameters of a magnetic resonance data acquisition that still has to occur.

### BACKGROUND OF THE INVENTION

1. Field of the Invention

The present invention generally relates to a method for controlling the execution of data acquisition sequences in individual tomography scans in a magnetic resonance tomography apparatus.

2. Description of the Prior Art

The magnetic resonance tomography is a method for the medical diagnosis which is primarily characterized by a high contrast resolving capability for soft tissue. Since the structures of the human body in this way can be represented in a detailed fashion, this imaging modality is in widespread use and has proven to be superior in many ways compared to other imaging modalities. The structures and organs of the human body are primarily identified by acquiring data representing slices of the human body. In order to completely acquire the region of the human body that is desired for a particular medical examination, it is necessary to image a number of such slices. It is also necessary to adapt the imaging of the individual slices to the particular conditions required for a specific medical examination. Therefore, it can be necessary to make a specific structure of the humane body more visible by a contrast medium. Another example is that it is possible to represent moving fluids and fluids in general such that they are rich in contrast, for example blood in the blood vessels. For this purpose, the appropriate parameters and settings, prior to the imaging of a specific tomogram, are also adjusted in a control program that controls a magnetic resonance tomography apparatus. Due to multiple medical examination possibilities enabled by magnetic resonance tomography, there is also a large number of different imaging possibilities for magnetic resonance tomography scans which are respectively optimized for the desired examination. An operator, therefore, must make a number of parameters and adjustments in a control program for the magnetic resonance tomography apparatus given a specific, desired medical examination and a specified region of the human body to be examined.

Furthermore, it can be necessary to multiply measure the same slice plane of the human body with different parameters. For example, this is the case when a specific area of the human body, such as the thorax or body torso, is to be initially imaged without using a contrast medium, and then after a contrast medium has been administered into a blood vessel. After the imaging, the results of the individual data acquisition sequences are initially present as datasets in the memory of the control computer of the magnetic resonance tomography apparatus. For example, if one or more additional slices of the same region of the human body are to be imaged in the magnetic resonance tomography apparatus, the operator must reenter the complete set of parameters by which the previous images were acquired. As a result, it is extremely time-consuming and complicated to undertake only one single additional imaging after a previously performed set of slice images has already been measured. In particular, it has previously not been possible to reconstruct the corresponding imaging parameters from the graphical representation as an image that is accessible to an operator and is displayed at the screen. Rather, it was necessary to completely reenter the imaging parameter set. Therefore, the entire measuring process was complicated and particularly time-intense.

German OS 43 10 993 discloses a magnetic resonance imaging method wherein a number of sequences each having at least one radio-frequency pulse and at least one magnetic gradient field act upon an examination area and wherein at least one magnetic resonance image is reconstructed from the thereby-acquired magnetic resonance signals. Furthermore, an arrangement for implementing this method is disclosed. An optimization can be achieved by analyzing the magnetic resonance signal or the magnetic resonance image by altering and the chronological curve of the radio-frequency pulses and/or of the magnetic gradient field, depending on the result of the analysis.

### SUMMARY OF THE INVENTION

An object of the present invention is to provide a method and a magnetic resonance tomography apparatus wherein, on the basis of a graphical representation of an already acquired image of the magnetic resonance tomography apparatus, it is possible, in a simple manner and without great outlay, to use the parameters associated with this nuclear magnetic resonance image for obtaining a subsequent new nuclear magnetic resonance image.

This object is inventively achieved in a method according to the invention wherein imaging parameters of already acquired magnetic resonance images, in a first step, are stored for preparing a subsequent magnetic resonance data acquisition session by using the already acquired magnetic resonance. In a second step, the acquired magnetic resonance images are subsequently or simultaneously graphically represented. In a third step, a selection possibility for one of the already acquired and graphically displayed magnetic resonance image is offered and graphically represented. In a further step, the imaging parameters of the magnetic resonance image selected by a user are automatically transferred for use as the imaging parameters of a subsequent magnetic resonance image to be acquired.

An advantage of the inventive method is that the user need not reenter the complete set of imaging parameters but, in an extremely simple and comfortable way, can re-execute a nuclear magnetic resonance image acquisition procedure by using the previous imaging parameters, or most of the previous imaging parameters, with subsequent adaptations and modifications. It is particularly advantageous that the complex operating sequence, which is required in known systems, is shortened, so that time is saved. On the basis of the selection by means of a graphical representation or the image formed by an already acquired dataset it is easily possible to select imaging parameters which have already proven to be particularly beneficial or advantageous for the desired medical examination or for the area of the human body to be represented. On the basis of the image, it can be simply determined whether the relevant structures or organs were able to be seen well in the image resulting from a previously performed data acquisition by means of the stored imaging parameters, and it can thus be simply determined whether the use of these imaging parameters is beneficial for a further measurement.

In an embodiment an automatic transfer of the imaging parameters occurs after a dataset (image) has been selected and clicked on by a mouse cursor for subsequent use in a series of measurements.

Therefore, the selection can occur in a simple manner. In particular, this type of selection, referred to as “drag and drop”, is known in, multiple software applications, so that a user can intuitively proceed. An object, the graphical image in this case, is marked by clicking and the selection occurs by pulling, i.e., a duplicate of the graphical image is pulled into the series of measurements by pressing the mouse button and the mouse pointer on the graphical image. Therefore, the operator must perform only two actions, and these two actions are interpreted easily and without much thinking to achieve transferring the imaging parameters for a future measurement in the series of measurements.

The above object also is achieved in a magnetic resonance tomography apparatus according to the invention having a control computer programmed to execute the above-described method. The described method is particularly advantageous for a magnetic resonance tomography apparatus since a large number of parameters must be adjusted to operate this examination device.

### DESCRIPTION OF THE DRAWINGS

FIG. 1 schematically shows a magnetic resonance tomography apparatus operable in accordance with the invention.

FIG. 2 shows a screen section of a control computer of an inventive magnetic resonance tomography apparatus in a schematic and simplified manner.

### DESCRIPTION OF THE PREFERRED EMBODIMENTS

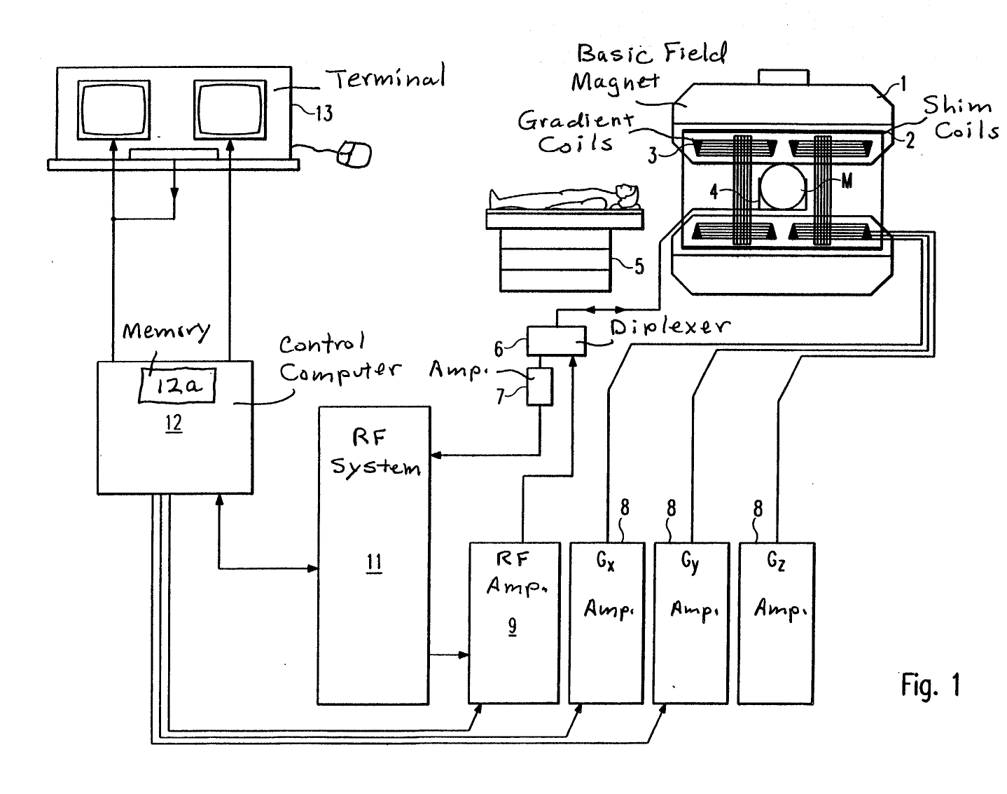

FIG. 1 schematically shows a magnetic resonance tomography apparatus for generating a magnetic resonance image of a subject, for executing the inventive method. The structure of the magnetic resonance tomography apparatus corresponds to the structure of a traditional magnetic resonance tomography apparatus. A basic magnetic field magnet 1 generates a chronologically constant strong magnetic field for the polarization (orientation) of the nuclear spins in the examination area of a subject, such as a part of a human body to be examined. The high homogeneity of the basic magnetic field required for the nuclear magnetic resonance measuring is defined in a spherical measuring volume M. The person to be examined, on a support 5 that can be moved into the magnetic resonance tomography apparatus, is introduced into this measuring volume M such that the parts of the human body to be examined are situated in the measuring volume M. In order to meet the homogeneity requirements with respect to the magnetic field and for eliminating time invariable influences, shim plates composed of ferromagnetic material are attached at appropriate locations. Shim coils 2 eliminate time variable influences. A cylindrical gradient coil system 3 composed of three sub-windings is inserted into the basic field magnet 1. Respective amplifiers 8 provide the sub-winding with current for generating respective linear gradient field in the directions of a Cartesian coordinate system. The first sub-winding of the gradient field system 3 generates a gradient Gₓ in the x-direction, the second sub-winding generates a gradient G_{y} in the y-direction and the third sub-winding generates a gradient G_{z} in the z-direction. On the basis of the gradient fields, the volume which is to be measured is given a selected resolution by being divided into voxels, each voxel corresponding to a point of the subsequent image. A radio-frequency antenna 4 is situated within the gradient field system 3, which converts the radio-frequency pulses emitted by a radio-frequency power amplifier 9 via a transmitter-receiver diplexer 6 into a magnetic alternating field for exciting the nuclei and for orienting the nuclear spins of the subject to be examined, or the region of the subject to be examined. The radio-frequency antenna 4 also converts the alternating field proceeding from the precessing nuclear spins (usually echo signals caused by a pulse sequence composed of one or more radio-frequency pulses and one or more gradient pulses) into a voltage that is supplied to a radio-frequency system 11 via the transmitter-receiver diplexer 6 and via an amplifier 7.

An image is generated in a known manner in a control computer 12 from the thus acquired measuring data. The administration of the image data and the imaging parameters required for the sequence of the individual measurements also occurs in the control computer 12. The combined effect of the gradient field system and the radio-frequency system 11 is controlled by these imaging parameters. In particular, the control computer 12 controls the sequence, i.e. the chronologically correct switching of the gradients, the sending of the radio-frequency pulses with a defined phase and amplitude, as well as the reception of the nuclear magnetic resonance signals. The selection of corresponding sets of imaging parameters for generating a magnetic resonance image and the representation of the generated magnetic resonance image also occur via the control computer 12 which has a terminal 13, a keyboard and mouse one or more screens.

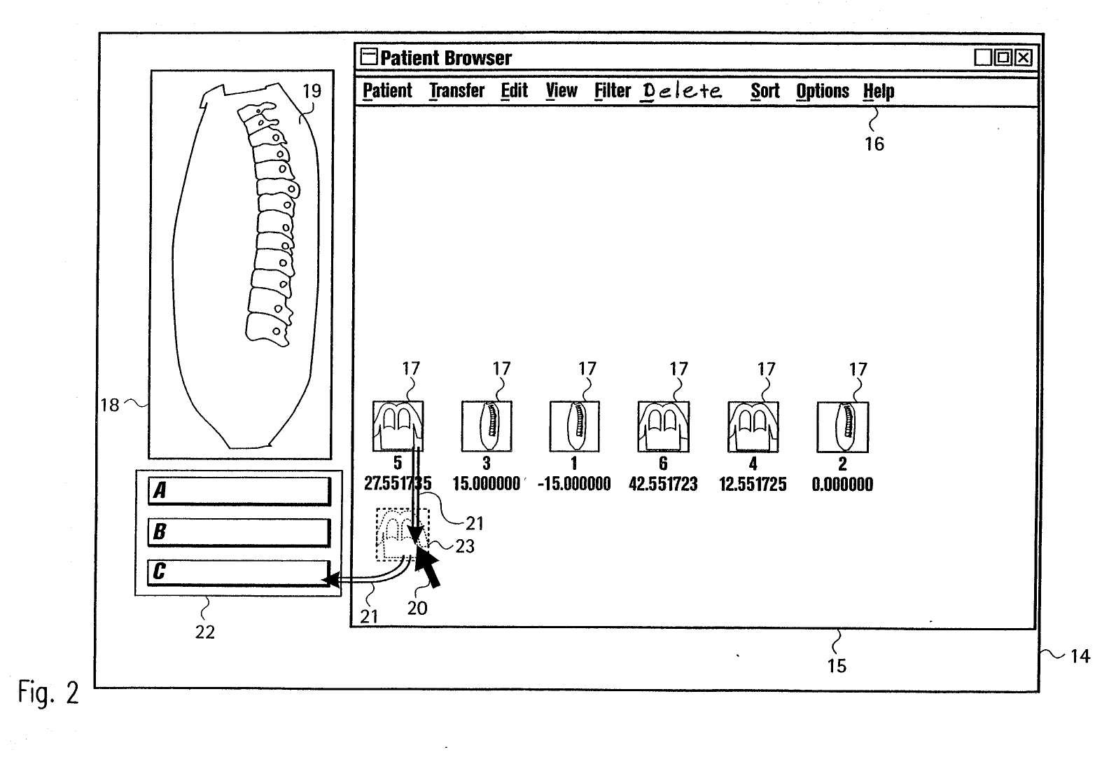

In a simplified manner, FIG. 2 schematically shows a screen section 14 of the control computer 12 in FIG. 1. A what is referred to as window 15 is represented therein as an element of a graphical user interface. Different possibilities for selecting pull-down menus are arranged in a menu bar 16. In addition, the window 15 shown in the exemplary embodiment of the invention, in the lower region, contains graphical symbols 17 for the already acquired images of magnetic resonance data acquisitions, which have been stored in a memory 12a in, or accessible by, the control computer 12. These are fashioned as reduced versions of the magnetic resonance images. The different images, six in this case, are characterized by a respectively allocated numbers and, for example, by a designation of the slice plane. A current image 18 is displayed at the screen section 14 next to the window 15. As an example it shows a longitudinal section through a human torso wherein the spine 19 can be recognized as a. In a symbolic representation of the series of measurements 22, the adjustments and parameters of the current image are represented by a switching area A and the adjustment and parameters of measurements to be performed in the series of measurements are represented by switching areas B and C, below the current image 18.

The adjustments and parameters represented by switching areas B, C, therefore, are for further data acquisitions still have to be performed, respectively for further images in the series. According to the inventive method, a selection possibility—to select the imaging parameters of a measurement that has already been performed, six in this case, is offered to the user via the graphical symbols 17. For example, the imaging parameters of the magnetic resonance pickup, which corresponds to the graphical symbol 17 having the indenture number 1 (at the far left in this case), are to be selected. The user of the control program 12, via a mouse and a mouse pointer 20, can now click on the graphical symbol having number 1 and, while holding down a suitable button determined on the mouse, drag symbol 17 to the switching surfaces B, C for measurements that still have to be performed. This is shown by the double arrows 21 and by a dragged symbol 23 held by the mouse pointer 7, so that the symbol 23 is moved from the position of the original graphical symbol 17 having the indenture number 1 to the third switching surface C. If the mouse button that was pressed is released, the imaging parameters of the magnetic resonance pickup, for which the selected graphical symbol 17 stands, are automatically assumed for the measurement that still has to be performed. For example, this can occur by starting a subroutine after the button has been released when the mouse pointer is situated above a switching surface C. This subroutine loads the stored imaging parameters of the selected image as a copy from the memory and transfers them, via an interface, to the program parts in which the imaging parameters are entered.

Alternatively, a new measurement to be performed, with a further symbol, can be integrated directly into the series 22 of measurements that still have to be performed and can be generated. A further symbol and therefore another measurement is generated when the button in the area of A, B or C is released.

On the basis of the described method and a magnetic resonance tomography apparatus using the described method, it is possible for a user, in an intuitive and simple manner, to use the measuring values and parameters of an image, which has already been performed, for a new nuclear magnetic resonance image. This is considerably simpler and saves time since acquisition sequences must normally be prepared with only a few parameters or parameters that are not modified.

Although modifications and changes may be suggested by those skilled in the art, it is in the intention of the inventors to embody within the patent warranted hereon all changes and modifications as reasonably and properly come within the scope of their contribution to the art.

We claim as our invention:

1. A method for preparing a magnetic resonance data acquisition, comprising the steps of:
storing imaging parameters respectively for previously-obtained magnetic resonance data acquisitions;
graphically representing said previously-obtained magnetic resonance data acquisitions in a graphics display;
making a selection in said graphics display of one of the graphically represented, previously-obtained magnetic resonance data acquisitions; and
automatically transferring the imaging parameters respectively associated with the selected, graphically represented, previously obtained magnetic resonance data acquisition for use as imaging parameters obtaining a subsequent magnetic resonance data acquisition.

2. A method as claimed in claim 1 comprising using a mouse-controlled cursor to select and drag the representation in said graphics display of said selected, graphically represented, previously obtained magnetic resonance data acquisition to a region on said graphics display for entering imaging parameters for said subsequent magnetic resonance data acquisition and automatically entering the imaging parameters associated with said selected, graphically represented previously obtained magnetic resonance data acquisition into said region.

3. A computer software product loadable into a memory for preparing a magnetic resonance data acquisition in a magnetic resonance tomography system for causing storage of imaging parameters respectively for previously-obtained magnetic resonance data acquisitions, graphically representing said previously-obtained magnetic resonance data acquisitions in a graphics display, allowing a selection in said graphics display of one of the graphically represented, previously-obtained magnetic resonance data acquisitions, and automatically transferring the imaging parameters respectively associated with the selected, graphically represented, previously obtained magnetic resonance data acquisition for use in obtaining a subsequent magnetic resonance data acquisition.

4. A computer software product as claimed in claim 3 allowing use of a mouse-controlled cursor to select and drag a representation in said graphics display of said selected, graphically represented previously obtained magnetic resonance data acquisition to a region on said graphics display for entering imaging parameters for said subsequent magnetic resonance data acquisition and causing automatic entry of the imaging parameters associated with said selected, graphically represented previously obtained magnetic resonance data acquisition into said region.

5. A magnetic resonance tomography system comprising:
an MR scanner operated by a control unit to obtain magnetic resonance data acquisitions, said control unit being connected to a terminal;
a memory accessible by said control unit in which imaging parameters respectively for previously-obtained magnetic resonance data acquisitions are stored;
said control unit graphically representing said previously-obtained magnetic resonance data acquisitions in a graphics display at said terminal;
said terminal allowing a selection in said graphics display of one of the graphically represented, previously-obtained magnetic resonance data acquisitions; and
said control unit automatically transferring the imaging parameters respectively associated with the selected, graphically represented, previously obtained magnetic resonance data acquisition for use in obtaining a subsequent magnetic resonance data acquisition.

6. A magnetic resonance tomography system as claimed in claim 5 wherein said terminal has a mouse, and wherein said terminal allows a cursor controlled by said mouse to select and drag a representation in said graphics display of said selected, graphically represented, previously obtained magnetic resonance data acquisition to a region on said graphics display for entering imaging parameters for said subsequent magnetic resonance data acquisition and wherein said control unit automatically enters the imaging parameters associated with said selected, graphically represented, previously obtained magnetic resonance data acquisition into said region.
